(19) United States
(12) Patent Application Publication
(10) Pub. No.: US 2024/0302259 A1
(43) Pub. Date: Sep. 12, 2024

(54) METHOD AND SYSTEM FOR PREDICTING PILE TIP RESISTANCE BASED ON DAMAGE CONSTITUTIVE MODEL OF MUDSTONE

(71) Applicant: Qingdao University of Technology, Qingdao (CN)

(72) Inventors: Nan YAN, Qingdao (CN); Xiaoyu BAI, Qingdao (CN); Jichao YIN, Qingdao (CN); Dongdong MA, Qingdao (CN); Yamei ZHANG, Qingdao (CN); Shaoshuai XU, Qingdao (CN); Yongliang XU, Qingdao (CN); Guangliang ZHANG, Qingdao (CN); Chenglu LV, Qingdao (CN); Jiashan WANG, Qingdao (CN); Zeqiang XU, Qingdao (CN); Haojie DIAO, Qingdao (CN)

(21) Appl. No.: 18/578,801

(22) PCT Filed: Jun. 2, 2023
(86) PCT No.: PCT/CN2023/097923

§ 371 Date: Jan. 12, 2024

(30) Foreign Application Priority Data
Jun. 23, 2022 (CN) 202210718270.0

(51) Int. Cl. G01N 3/60; G01N 3/08; G01N 33/24; G06F 30/20; G06F 119/14

(52) U.S. Cl. CPC G01N 3/60; G01N 3/08; G01N 33/24; G06F 30/20; G06F 2119/14

(57) ABSTRACT

The present disclosure provides a method and system for predicting pile tip resistance based on a damage constitutive model of mudstone and relates to the technical field of geotechnical engineering. In view of the existing problem of difficult calculation of quantitative analysis on the bearing performance of a dynamic driven pile of a mudstone foundation and the damage characteristics of mudstone around the pile, a damage constitutive model of mudstone with a damage variable D determined based on a stress state and associated with a rock failure criterion is established; model parameters are determined based on results of a triaxial test; a relationship between a critical damage variable and a confining pressure is established; and the damage characteristics of the mudstone around the dynamic driven pile are analyzed, whereby pile tip resistance is calculated. The requirements of quantitative analysis on the damage characteristics of a pile tip are met.

This patent application claims the benefit and priority of Chinese Patent Application No. 202210718270.0 filed with the China National Intellectual Property Administration on Jun. 23, 2022, and entitled “METHOD AND SYSTEM FOR PREDICTING PILE TIP RESISTANCE BASED ON DAMAGE CONSTITUTIVE MODEL OF MUDSTONE”, the disclosure of which is incorporated by reference herein in its entirety as part of the present application.

### TECHNICAL FIELD

The present disclosure relates to the technical field of geotechnical engineering, and in particular, to a method and system for predicting pile tip resistance based on a damage constitutive model of mudstone.

### BACKGROUND

Rock is a heterogeneous anisotropic geological material that contains sensitive defects such as fractures and cavities and is complex in constitutive relationship, and a constitutive model of rock and engineering application of the constitutive model are always research emphasis in the field of geotechnical engineering. Soft rock, especially mudstone, has a constitutive property and is a common geotechnical engineering material. A force bearing process of mudstone is an internal defect development process, and therefore, the mudstone itself has initial damage. Moreover, such a process of the internal defect developing progressively and resulting in degradation of mechanical properties of the mudstone is irreversible, and this mechanical behavior does not belong to a strict elasticoplastic deformation scope. Therefore, a description based on the elastic-plastic theory cannot completely reflect deformation characteristics of the mudstone. Damage mechanics is a theory established to study the damage evolution and destruction process of a material under the action of a load, and therefore, introducing a damage theory to study the damage evolution and mechanical properties of the mudstone is of important theoretical and practical significance.

At present, in research about a mudstone damage model, triaxial test mudstone on which the establishment of the mudstone damage model and the calculation of parameters are based is mainly taken from high stress deep mudstone in mineral engineering, tunnel engineering, and the like. It is insufficient in research on mudstone around a dynamic driven pile. Due to special engineering properties of the mudstone, such as being easily disturbed and softened by water, the bearing capacity of a pile foundation in a mudstone foundation is frequently abnormal. In a dynamic pile sinking process, soil around a pile will produce soil response. For the mudstone foundation, a dynamic driving process of a precast pile is accompanied with secondary damage. The initial damage is coupled with the dynamic pile driving damage to exert an influence on the bearing characteristics of the mudstone foundation, often leading to an abnormal phenomenon that the vertical compressive bearing capacity of a single pile does not meet the design requirement after engineering piles pass acceptance inspection, with major features manifesting as small pile tip force and significant pile tip settlement displacement. In view of this problem, the prior art CN202110681095.8 discloses a method for establishing a damage model of a cemented filling body containing initial damage. In this method which is restricted, it is difficult to determine an initial damage threshold after mudstone around a pile undergoes dynamic pile driving damage. CN202010825183.6 discloses a method for estimating the single-pile bearing capacity of a threaded pile, where when calculating pile tip resistance, not only may soil parameters and pile parameters such as a pile diameter, a thread pitch, a thread height, and a thread width be required, but also on-site construction parameters and static test data are needed, and calculation is complicated and restricted to a type of the threaded pile. Therefore, it is hard to meet the requirements of quantitative analysis on the bearing performance of the dynamic driven pile of the mudstone foundation and the damage characteristics of the mudstone around the pile.

### SUMMARY

The present disclosure provides a method and system for predicting pile tip resistance based on a damage constitutive model of mudstone. A statistical damage constitutive model of mudstone with a damage variable D determined based on a stress state and associated with a rock failure criterion is established: model parameters are determined based on results of a triaxial test: a relationship between a critical damage variable and a confining pressure is established; and the damage characteristics of mudstone around a dynamic driven pile are analyzed, whereby pile tip resistance is calculated. The requirements of quantitative analysis on damage characteristics of a pile tip are met.

To achieve the above objective, the embodiments of the present disclosure provide the following technical solutions.

The present disclosure provides a method for predicting pile tip resistance based on a damage constitutive model of mudstone, including:
obtaining mechanical parameters of mudstone according to a triaxial compression test, and defining a damage variable;
according to a strain equivalence principle, establishing a relationship of a stress and a strain with the damage variable, establishing a damage evolution equation in combination with the mechanical parameters of the mudstone, and fitting a relationship equation of the damage variable, a deviatoric stress, and a confining pressure;
establishing a damage constitutive model of mudstone, and calculating parameters of the damage constitutive model in combination with results of a triaxial test to obtain a correspondence between a confining pressure and a stress peak; and
obtaining force data of the mudstone at a pile tip, and substituting the force data as a confining pressure into the correspondence to obtain pile tip resistance.

Alternatively, the method may further include: calculating an elasticity modulus and a shear strength indicator to obtain the mechanical parameters of the mudstone.

Alternatively, the method may further include: discretizing a material into a plurality of primitives, where the damage variable is a ratio of a cross-sectional area of damage after the material is damaged to an initial cross-sectional area of the mudstone, and a primitive strength is assumed to comply with Weibull distribution.

Alternatively, the method may specifically include: calculating the damage variable based on data of a conventional triaxial test, and fitting the relationship equation of the damage variable, a deviatoric stress, and a confining pressure.

Alternatively, the method may specifically include: obtaining, according to the strain equivalence principle, a mudstone damage stress-strain relationship based on Weibull distribution as the damage constitutive model of mudstone, and optimizing the damage constitutive model of mudstone.

Alternatively, the calculating parameters of the damage constitutive model may include the following steps:
recording a stress, a strain, and a confining pressure corresponding to a peak point after a stress-strain curve of mudstone under a confining pressure reaches an ultimate strength; and
calculating a mudstone primitive strength corresponding to the peak point based on the results of the triaxial test, and calculating the parameters of the damage constitutive model.

Alternatively, the method may further include: substituting the obtained parameters of the damage constitutive model into the damage constitutive model, calculating the damage variable and the strain according to a loading grade of the triaxial test, and comparing the calculated damage variable and strain with the results of the triaxial test for verification.

Alternatively, the method may specifically include: fitting a function relationship between a confining pressure and a peak point by means of the triaxial test and damage analysis calculation to obtain the correspondence between a confining pressure and a stress peak.

Alternatively, the method may specifically include: obtaining pile parameters and a unit weight of a rock-soil body, and substituting a horizontal soil pressure of the mudstone at the pile tip as a confining pressure into the correspondence to obtain a corresponding vertical destructive force which is equivalent to a concentrated load as the pile tip resistance.

The present disclosure further provides a system for predicting pile tip resistance based on a damage constitutive model of mudstone, including:
a parameter obtaining module configured to: obtain mechanical parameters of mudstone according to a triaxial compression test, and define a damage variable;
a fitting module configured to: according to a strain equivalence principle, establish a relationship of a stress and a strain with the damage variable, establish a damage evolution equation in combination with the mechanical parameters of the mudstone, and fit a relationship equation of the damage variable, a deviatoric stress, and a confining pressure;
a modeling module configured to: establish a damage constitutive model of mudstone, and calculate parameters of the damage constitutive model in combination with results of a triaxial test to obtain a correspondence between a confining pressure and a stress peak; and
a data output module configured to: obtain force data of the mudstone at a pile tip, and substitute the force data as a confining pressure into the correspondence to obtain pile tip resistance.

Compared with the prior art, the present disclosure has the following advantages and positive effects.

(1) In view of the existing problem of difficult calculation of quantitative analysis on the bearing performance of a dynamic driven pile of a mudstone foundation and the damage characteristics of mudstone around the pile, a statistical damage constitutive model of mudstone with a damage variable D determined based on a stress state and associated with a rock failure criterion is established: model parameters are determined based on results of a triaxial test: a relationship between a critical damage variable and a confining pressure is established; and the damage characteristics of the mudstone around the dynamic driven pile are analyzed, whereby pile tip resistance is calculated. The requirements of quantitative analysis on the damage characteristics of a pile tip are met.

(2) The damage constitutive model is verified in combination with the triaxial test: the damage variable D is calculated according to a loading grade of the triaxial test: a strain is then calculated from a stress, and a comparison diagram of model calculation results with a curve of the triaxial test is plotted with an axial strain as x-axis and a deviatoric stress as y-axis. The accuracy of the novel damage constitutive model is verified.

### BRIEF DESCRIPTION OF THE DRAWINGS

To describe the technical solutions in the embodiments of the present disclosure or in the prior art more clearly, the accompanying drawings required in the embodiments will be briefly described below. Apparently, the accompanying drawings in the following description show merely some embodiments of the present disclosure, and other drawings can be derived from these accompanying drawings by those of ordinary skill in the art without creative efforts.

FIG. 1 is a flowchart of a damage constitutive model and a method for predicting pile tip resistance in Example 1 or 2 of the present disclosure;

FIG. 2 is a schematic diagram of a damage evolution curve in Example 1 or 2 of the present disclosure;

FIG. 3 is a D-q-σ₃ spatial scatter diagram in Example 1 or 2 of the present disclosure;

FIG. 4 is a relationship diagram of D-q-σ₃ fitted equation and a spatial curved surface in Example 1 or 2 of the present disclosure;

FIG. 5 is a comparison diagram of D-q-σ₃ fitted data vs test data in Example 1 or 2 of the present disclosure;

FIG. 6 is a comparison diagram of model calculation results vs a curve of a triaxial test in Example 1 or 2 of the present disclosure;

FIG. 7 is a relationship diagram of a confining pressure σ3 and a peak point σ_{1c} in Example 1 or 2 of the present disclosure;

FIG. 8 is a schematic diagram of a force state of mudstone at a pile tip in Example 1 or 2 of the present disclosure; and

FIG. 9 is a force diagram of a pile tip in an on-site test in Example 1 or 2 of the present disclosure.

### DETAILED DESCRIPTION OF THE EMBODIMENTS

The technical solutions in the embodiments of the present disclosure will be described below clearly and completely with reference to the accompanying drawings in the embodiments of the present disclosure. Apparently, the described embodiments are merely some rather than all of the embodiments of the present disclosure. All other embodiments derived from the embodiments in the present disclosure by a person of ordinary skill in the art without creative efforts shall fall within the protection scope of the present disclosure.

### Example 1

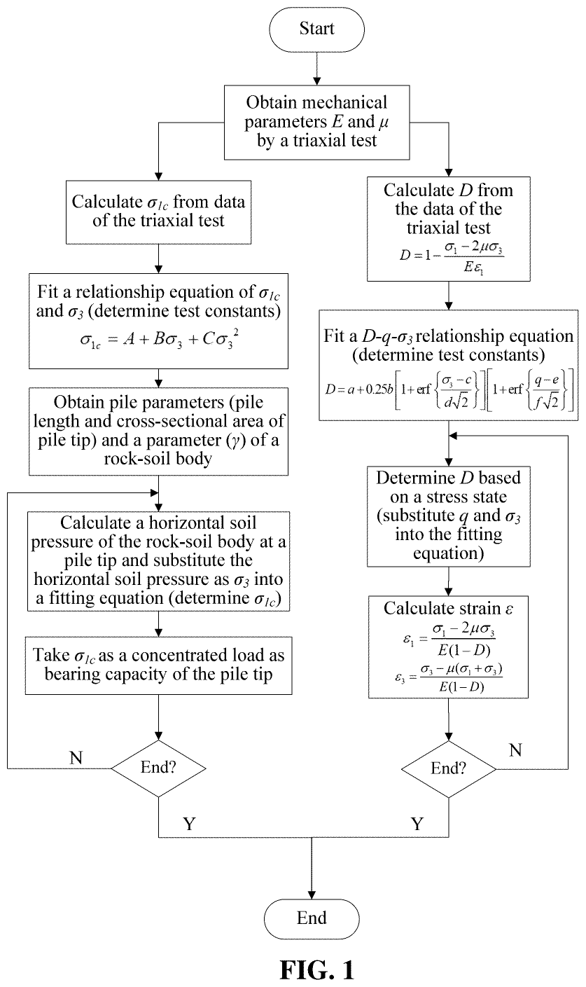

In a typical embodiment of the present disclosure, as shown in FIG. 1 to FIG. 9, there is provided a method for predicting pile tip resistance based on a damage constitutive model of mudstone.

As shown in FIG. 1, a statistical damage constitutive model of mudstone with a damage variable D determined based on a stress state and associated with a rock failure criterion is established: model parameters are determined based on results of a triaxial test: a relationship between a critical damage variable and a confining pressure is established: the damage characteristics of mudstone around a dynamic driven pile are analyzed: a function relationship formula between a confining pressure σ₃ and a peak point σ_{1c} is fitted: a horizontal soil pressure of the mudstone at a pile tip is used as a confining pressure σ₃ to calculate pile tip resistance, especially tip resistance of a precast pile of a mudstone foundation, and the damage constitutive model is verified in combination with laboratory and field tests.

With reference to FIG. 1, the damage constitutive model of mudstone and the method for predicting pile tip resistance include the following contents.

1. Mechanical parameters are obtained according to a triaxial compression test. The triaxial compression test is conducted on mudstone to obtain the mechanical parameters of the mudstone, and elasticity moduli E and μ and shear strength indicators c and φ are calculated.

2. It is assumed that a rock primitive strength F complies with Weibull distribution. The rock medium is discretized and may be regarded as different primitives. A primitive has two states: damaged and undamaged. Differences in defects and properties of a material are reflected by a sequential order of independent primitives being damaged. The accumulation of numbers of damaged primitives results in changes in properties of the material, manifested as degradation of mechanical properties on the macro level.

It is assumed that a rock primitive strength F complies with Weibull distribution, and a probability density function thereof is expressed as:

$$P ( F ) = \frac{m}{{F}_{0}} {( \frac{F}{{F}_{0}} )}^{m - 1} exp [ - {( \frac{F}{{F}_{0}} )}^{m} ] \qquad ( 1 )$$

where F represents the rock primitive strength: m and F₀ represent Weibull parameters, i.e., subsequent model parameters.

3. A damage variable D is defined. Damage is an intuitive form with a certain geometrical shape such as a microcrack and a microdefect within a material. Assuming that an initial cross-sectional area of a force bearing object is A and a cross-sectional area of damage after the force bearing object is damaged is A″, a net cross-sectional area is expressed as:

$${A}^{′} = A - {A}^{″} \qquad ( 2 )$$

In this case, the damage variable is expressed as:

$$D = \frac{{A}^{″}}{A} = \frac{A - {A}^{′}}{A} \qquad ( 3 )$$

where D=0 corresponds to an undamaged state; 0<D<1 corresponds to a partially damaged state; and D=1 corresponds to a completely damaged state. In addition, σ=F/A is a nominal stress of a cross section; and σ′=F/A′ is an effective stress, namely a net stress, of the cross section.

4. According to a strain equivalence principle, a relationship of a stress and a strain with the damage variable is established. The strain equivalence principle is described as a strain induced by a stress (nominal stress) acting on a damaged material being equivalent to a strain induced by an effective stress (net stress) acting on an undamaged material having same geometric dimensions, which is expressed as:

$$ε = \frac{σ}{{E}^{′}} = \frac{{σ}^{′}}{E} = \frac{σ}{( 1 - D ) E} \qquad ( 4 )$$

where E and E′ represent elasticity moduli of the undamaged material and the damaged material. According to the strain equivalence principle, a constitutive relationship of the damaged material may be expressed by the nominal stress of the undamaged material.

5. A damage evolution equation is established. An evolution equation of the damage variable may be obtained by using a distribution density function:

$$D = {\int }_{0}^{F} P ( x ) dx = 1 - exp [ - {( \frac{F}{{F}_{0}} )}^{m} ] \qquad ( 5 )$$

According to existing research, it is assumed that the general formula of the rock failure criterion is as follows:

f(σ′)−k₀=0 (6)
where k₀ represents a constant related to the cohesion and an internal friction angle of a material.

For different failure criteria, F may be expressed in different forms. In consideration of the extensive application scope, a primitive strength F based on Mohr-Coulomb rock strength theory is adopted. A Mohr-Coulomb rock strength criterion is expressed as:

$${σ}_{1}^{′} = \frac{1 + sin φ}{1 - sin φ} , \qquad ( 7 )$$

$${σ}_{3}^{′} = \frac{2 c cos φ}{1 - 2 sin φ}$$

where c and φ represent the cohesion and an internal friction angle of rock, respectively. Thus, the rock primitive strength is determined as:

$$F = {σ}_{1}^{′} - {ασ}_{3}^{′} \qquad ( 8 )$$

where α=(1−sin φ)/(1+sin φ).

6. A damage constitutive model is established. Based on strain equivalence hypothesis, the following damage constitutive relationship of mudstone may be established:

$$[ {σ}^{′} ] = [ σ ] / ( 1 - D ) = [ C ] [ ε ] / ( 1 - D ) \qquad ( 9 )$$

where [C] represents an elasticity matrix of the mudstone material: [σ′] represents an effective stress matrix: [σ] represents a nominal stress matrix: [ε] represents a strain matrix; and D represents the damage variable of the mudstone. The following formula may be derived from the above formula:

$${ε}_{1} = [ {σ}_{1}^{′} - μ ( {σ}_{2}^{′} + {σ}_{3}^{′} ) ] / E \qquad ( 10 )$$

$$or ,$$

$$D = [ {σ}_{1}^{′} - μ ( {σ}_{2} + {σ}_{3} ) ] / ( E {ε}_{1} ) \qquad ( 11 )$$

where σ₁′=σ₁′/(1−D): σ₂′=σ₂′/(1−D); and σ₃′=σ₃′/(1−D), from which the following formula may be derived:

$${σ}_{1}^{′} = {σ}_{1} / ( 1 - D ) F = ( {σ}_{1} - {ασ}_{3} ) E {ε}_{1} / [ {σ}_{1} - μ ( {σ}_{2} + {σ}_{3} ) ] \qquad ( 12 )$$

By substituting the formula (12) into the formula (9), a damage stress-strain relationship of mudstone damage based on Weibull distribution, i.e., the damage constitutive relationship, may be derived:

$${σ}_{1} = E {ε}_{1} ( 1 - D ) + μ ( {σ}_{2} + {σ}_{3} ) = E {ε}_{1} exp [ - {( F / {F}_{0} )}^{m} ] + μ ( {σ}_{2} + {σ}_{3} ) \qquad ( 13 - a )$$

$${σ}_{2} = E {ε}_{2} ( 1 - D ) + μ ( {σ}_{1} + {σ}_{3} ) = E {ε}_{2} exp [ - {( F / {F}_{0} )}^{m} ] + μ ( {σ}_{1} + {σ}_{3} ) \qquad ( 13 - b )$$

$${σ}_{3} = E {ε}_{3} ( 1 - D ) + μ ( {σ}_{1} + {σ}_{2} ) = E {ε}_{3} exp [ - {( F / {F}_{0} )}^{m} ] + μ ( {σ}_{1} + {σ}_{2} ) \qquad ( 13 - c )$$

For a conventional triaxial test, σ₂=σ₃, ε₂=ε₃, and the above formula may be simplified as:

$${σ}_{1} = E {ε}_{1} exp [ - {( F / {F}_{0} )}^{m} ] + 2 {μσ}_{3} \qquad ( 14 )$$

To better describe the damage evolution of mudstone around a pile and enable use thereof in analysis on the bearing capacity of the pile of the mudstone foundation, the modeling method may be optimized. The data of the triaxial test on the mudstone is fitted according to the concept of the damage theory. A relationship of the damage variable with a stress and a strain is directly established. The damage variable D is directly determined from a stress state. The calculation process of a damage degree is simplified. Thus, a new damage constitutive model is established. An optimized damage model is established still based on the basic concept and definition of the damage theory described above, and the specific process is as follows.

(1) The damage variable D is defined according to the formula (3).

(2) According to the formula (4) of the Lemaitre's strain equivalence principle, the relationship of a stress and a strain with the damage variable is established:

$$ε = \frac{σ}{( 1 - D ) E} \qquad ( 15 )$$

(3) The damage evolution equation is established. According to the formula (13-a), the following formula may be derived under a conventional triaxial condition:

$$D = 1 - \frac{{σ}_{1} - 2 μ {σ}_{3}}{E {ε}_{1}} \qquad ( 16 )$$

(4) According to the data of the conventional triaxial test, a relationship equation regarding the damage variable D, a deviatoric stress q, and a confining pressure σ₃ is fitted. When nonlinear fitting is performed on the data, due to the uncertainty of a relationship between variables, feature analysis needs to be performed on test data, and then an equation of a corresponding variation trend is selected for fitting. To seek for an optimal fitting function and fitting parameters, different models need to be compared on goodness of fit. Variance analysis is performed with a residual, a determination coefficient, and a weighted chi-squared test coefficient, and quantitative evaluation on a fitting result may be achieved in combination with Akaike information criterion (AIC) and the like. Finally, a Gaussian function (Gausscum equation) is selected for fitting, and the equation is as follows:

$$D = a + 0.25 b [ 1 + erf \{ \frac{{σ}_{3} - c}{d \sqrt{2}} \} ] [ 1 + erf \{ \frac{q - e}{f \sqrt{2}} \} ] \qquad ( 17 )$$

where a, b, c, d, e, and f represent test constants.

(5) A constitutive relationship equation is established. According to the formula (13), the following formulas may be derived under a conventional triaxial test condition:

$${ε}_{1} = \frac{{σ}_{1} - 2 μ {σ}_{3}}{E ( 1 - D )} \qquad ( 18 - a )$$

$${ε}_{3} = \frac{{σ}_{3} - μ ( {σ}_{1} + {σ}_{3} )}{E ( 1 - D )} \qquad ( 18 - b )$$

This model is based on the conventional triaxial test with no consideration of an intermediate principal stress. The intermediate principal stress has little influence on soil strength, and the influence of the intermediate principal stress σ₂ may be neglected.

7. Damage model parameters are determined. A descending segment (softening) occurs after a stress-strain curve of mudstone under a certain confining pressure reaches an ultimate strength, and therefore, there is an extreme value. A stress and a strain corresponding to a peak point are recorded as σ_{1c} and ε_{1c}, respectively. The mudstone primitive strength F_{c} corresponding to the peak point is calculated according to the results of the triaxial test:

$${F}_{c} = \frac{[ α ( {σ}_{1 c} + 2 {σ}_{3} ) + \frac{{σ}_{1 c} - {σ}_{3}}{\sqrt{3}} ] E {ε}_{1 c}}{{σ}_{1 c} - 2 μ {σ}_{3}} \qquad ( 19 )$$

The model parameters m and F₀ are calculated by the following formula:

$$m = \frac{1}{ln [ \frac{E {ε}_{1 c}}{{σ}_{1 c} - 2 μ {σ}_{3}} ]} ; {F}_{0} = {F}_{c} {m}^{\frac{1}{m}} \qquad ( 20 )$$

8. A formula of a function relationship of a confining pressure σ₃ and a peak point σ_{1c} is fitted. By the triaxial test and damage analysis calculation, a relationship equation of a confining pressure σ₃ and a peak point σ_{1c} is obtained by fitting the function relationship therebetween using the method of least squares:

σ_{1c}=A+Bσ₃+Cσ₃² (21)
where A, B, and C represent test constants.

9. Pile tip resistance is calculated. A force condition of mudstone at a pile tip is investigated. A force state of the mudstone at the pile tip is similar to a force state of the mudstone in the triaxial test. Pile parameters (a pile length and a cross-sectional area of the pile tip) and a unit weight γ of a rock-soil body are obtained. A horizontal soil pressure of the mudstone at the pile tip is substituted as the confining pressure σ₃ into the formula (21) to obtain a corresponding vertical destructive force σ_{1c} which is equivalent to a concentrated load as the pile tip resistance.

Specifically, in this embodiment, the method for predicting pile tip resistance based on a damage constitutive model of mudstone is described in detail with reference to FIG. 1 to FIG. 9.

1. Mechanical parameters are obtained according to a triaxial compression test. Intermediary weathered mudstone at a pile tip affected by pile driving is obtained by drilling. A core barrel has a diameter of 73 mm and the obtained mudstone test sample has a diameter of 50 mm. An experimental apparatus is a high-pressure low-temperature static triaxial test system for a hydrate. The apparatus is manufactured by GDS Instruments, and the model of the apparatus is ETAS with a maximum confining pressure of 32 MPa and a maximum axial force of 100 kN. A standard triaxial test can be conducted on the system, and a test confining pressure is set to 0.5 MPa, 1.0 MPa, 1.5 MPa, and 2.0 MPa. Elasticity moduli E and μ and shear strength indicators c and φ are calculated according to the results of the triaxial test on the mudstone. The calculation results of E are shown in Table 1. Poisson's ratio μ is 0.3; and calculated average values of the shear strength indicators are as follows: c=217.2 kPa and φ=21.6°.

TABLE 1
Table of Parameter E Determined According
to Triaxial Test on Mudstone

| [symbol] /kPa | [symbol] | [symbol] /kPa | [symbol] /kPa | E/MPa |
|---|---|---|---|---|
| 500 | 0.0025 | 122 | 622 | 248.8 |
| 1000 | 0.0025 | 166 | 1166 | 466.4 |
| 1500 | 0.0025 | 235 | 1735 | 694.0 |
| 2000 | 0.0025 | 298 | 2298 | 919.2 |

[symbol]

indicates data missing or illegible when filed

2. It is assumed that a rock primitive strength F complies with Weibull distribution. The rock medium is discretized and may be regarded as different primitives. A primitive has two states: damaged and undamaged. Differences in defects and properties of a material are reflected by a sequential order of independent primitives being damaged. The accumulation of numbers of damaged primitives results in changes in properties of the material, manifested as degradation of mechanical properties on the macro level. It is assumed that the rock primitive strength F complies with Weibull distribution, and the probability density function thereof is expressed as:

$$P ( F ) = \frac{m}{{F}_{0}} {( \frac{F}{{F}_{0}} )}^{m - 1} exp [ - {( \frac{F}{{F}_{0}} )}^{m} ] \qquad ( 22 )$$

3. A damage variable D is defined. Damage is an intuitive form with a certain geometrical shape such as a microcrack and a microdefect within a material. Assuming that the initial cross-sectional area of a force bearing object is A and the cross-sectional area of damage after the force bearing object is damaged is A″, the net cross-sectional area is expressed as:

$${A}^{′} = A - {A}^{″} \qquad ( 23 )$$

In this case, the damage variable is expressed as:

$$D = \frac{{A}^{″}}{A} = \frac{A - {A}^{′}}{A} \qquad ( 24 )$$

4. According to the strain equivalence principle, a relationship of a stress and a strain with the damage variable is established. The strain equivalence principle is described as a strain induced by a stress (nominal stress) acting on a damaged material being equivalent to a strain induced by an effective stress (net stress) acting on an undamaged material having same geometric dimensions, which is expressed as:

$$ε = \frac{σ}{{E}^{′}} = \frac{{σ}^{′}}{E} = \frac{σ}{( 1 - D ) E} \qquad ( 25 )$$

5. A damage evolution equation is established. An evolution equation of the damage variable may be obtained by using a distribution density function:

$$D = {\int }_{0}^{F} P ( x ) dx = 1 - exp [ - {( \frac{F}{{F}_{0}} )}^{m} ] \qquad ( 26 )$$

According to existing research, it is assumed that the general formula of the rock failure criterion is as follows:

$$f ( {σ}^{′} ) - {k}_{0} = 0 \qquad ( 27 )$$

For different failure criteria, F may be expressed in different forms. In consideration of the extensive application scope, the primitive strength F based on Mohr-Coulomb rock strength theory is adopted. The Mohr-Coulomb rock strength criterion is expressed as:

$${σ}_{1}^{′} = \frac{1 + sin φ}{1 - sin φ} , {σ}_{3}^{′} = \frac{2 c cos φ}{1 - 2 sin φ} \qquad ( 28 )$$

where c and φ represent the cohesion and an internal friction angle of rock, respectively.

Thus, the rock primitive strength is determined as:

$$F = {σ}_{1}^{′} - {ασ}_{3}^{′} \qquad ( 29 )$$

where α=(1−sin φ)/(1+sin φ).

6. A damage constitutive model is established. Based on strain equivalence hypothesis, the following damage constitutive relationship of mudstone may be established:

$$[ {σ}^{′} ] = [ σ ] / ( 1 - D ) = [ C ] [ ε ] / ( 1 - D ) \qquad ( 30 )$$

The following formula may be derived from the above formula:

$${ε}_{1} = [ {σ}_{1}^{′} - μ ( {σ}_{2}^{′} + {σ}_{3}^{′} ) ] / E \qquad ( 31 )$$

$$or ,$$

$$D = [ {σ}_{1}^{′} - μ ( {σ}_{2} + {σ}_{3} ) ] / ( E {ε}_{1} ) \qquad ( 32 )$$

where σ₁′=σ₁′/(1−D): σ₂′=σ₂′/(1−D); and σ₃′=σ₃′/(1−D), from which the following formula may be derived:

$${σ}_{1}^{′} = {σ}_{1} / ( 1 - D ) F = ( {σ}_{1} - {ασ}_{3} ) E {ε}_{1} / [ {σ}_{1} - μ ( {σ}_{2} + {σ}_{3} ) ] \qquad ( 33 )$$

By substituting the formula (33) into the formula (30), a damage stress-strain relationship of mudstone damage based on Weibull distribution, i.e., the damage constitutive relationship, may be derived:

$${σ}_{1} = E {ε}_{1} ( 1 - D ) + μ ( {σ}_{2} + {σ}_{3} ) = E {ε}_{1} exp [ - {( F / {F}_{0} )}^{m} ] + μ ( {σ}_{2} + {σ}_{3} ) \qquad ( 34 - a )$$

$${σ}_{2} = E {ε}_{2} ( 1 - D ) + μ ( {σ}_{1} + {σ}_{3} ) = E {ε}_{2} exp [ - {( F / {F}_{0} )}^{m} ] + μ ( {σ}_{1} + {σ}_{3} ) \qquad ( 34 - b )$$

$${σ}_{3} = E {ε}_{3} ( 1 - D ) + μ ( {σ}_{1} + {σ}_{2} ) = E {ε}_{3} exp [ - {( F / {F}_{0} )}^{m} ] + μ ( {σ}_{1} + {σ}_{2} ) \qquad ( 34 - c )$$

For a conventional triaxial test, σ₂=σ₃, ε₂=ε₃, and the above formula may be simplified as:

$${σ}_{1} = E {ε}_{1} exp [ - {( F / {F}_{0} )}^{m} ] + 2 {μσ}_{3} \qquad ( 35 )$$

To better describe the damage evolution of mudstone around a pile and enable use thereof in analysis on the bearing capacity of the pile of the mudstone foundation, the modeling method may be optimized. The data of the triaxial test on the mudstone is fitted according to the concept of the damage theory. A relationship of the damage variable with a stress and a strain is directly established. The damage variable is directly determined from a stress state. The calculation process of a damage degree is simplified. Thus, a new damage constitutive model is established. An optimized damage model is established still based on the basic concept and definition of the damage theory described above, and the specific process is as follows:

(1) The damage variable D is defined according to the formula (24).

(2) According to the formula (25) of the Lemaitre's strain equivalence principle, the relationship of a stress and a strain with the damage variable is established:

$$ε = \frac{σ}{( 1 - D ) E} \qquad ( 36 )$$

(3) The damage evolution equation is established. According to the formula (34-a), the following formula may be derived under a conventional triaxial condition:

$$D = 1 - \frac{{σ}_{1} - 2 {μσ}_{3}}{E {ε}_{1}} \qquad ( 37 )$$

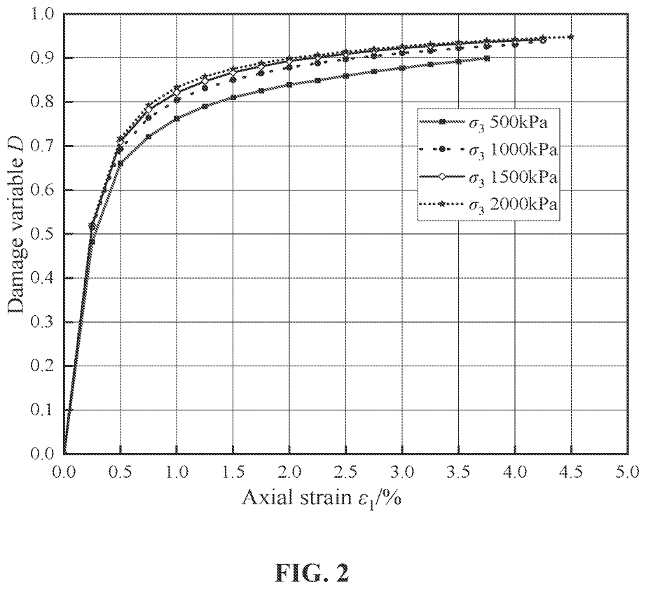

According to the results of a triaxial test on mudstone, the damage variable D and the axial strain ε₁ of the mudstone are calculated by the formula (37), and a damage evolution curve of mudstone is plotted with the axial strain &1 as x-axis and the damage variable D as y-axis, as shown in FIG. 2.

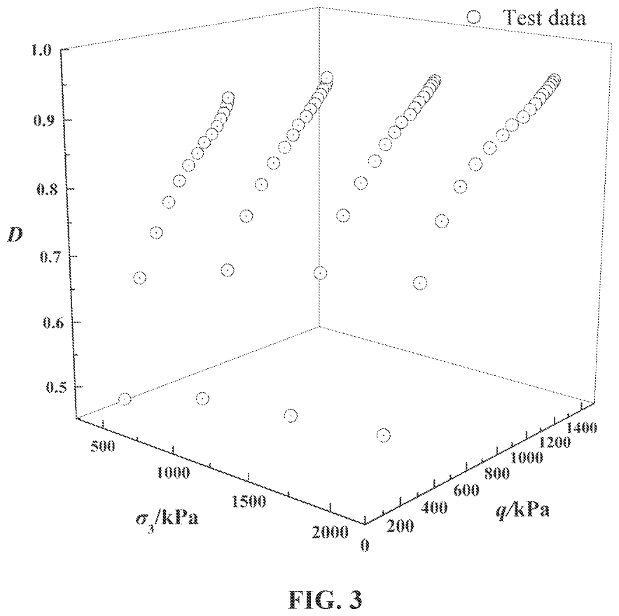

(4) The relationship equation of the damage variable D, a deviatoric stress q, and a confining pressure σ₃ is fitted. As shown in FIG. 3, a D-q-σ₃ spatial scatter diagram is plotted with the data of a conventional triaxial test, and nonlinear fitting is performed on the data according to spatial scatter features. When nonlinear fitting is performed on the data, due to the uncertainty of a relationship between variables, feature analysis needs to be performed on test data, and then an equation of a corresponding variation trend is selected for fitting. To seek for an optimal fitting function and fitting parameters, different models need to be compared on goodness of fit. The goodness of fit is determined by determining how much the fitted model fits actual data. However, the determination is not quantitative. Variance analysis is performed with a residual, a determination coefficient, and a weighted chi-squared test coefficient, and quantitative evaluation on a fitting result may be achieved in combination with the AIC and the like. Finally, as shown in Table 2. When applied to the variance analysis, the AIC may be expressed as:

$$AIC = n ln {\hat{σ}}^{2} + 2 p \qquad ( 38 )$$

where n represents a number of samples, namely a number of data points; represents a residual sum of squares; and p represents an independent parameter dimension in the model, namely a degree of freedom.

TABLE 2
Evaluation Table of Goodness of Fit

| | | | | | Fitting |
|---|---|---|---|---|---|
| Fitting | | | | | Evalua- |
| Method | RCS | R² | RSS | AIC | tion |
| Gausscum | 4.11 × 10⁻⁴ | 0.96545 | 0.02549 | −121.85442 | Good |
| equation | | | | | |
| Exponential | 9.24 × 10⁻⁴ | 0.92236 | 0.05822 | −64.51626 | Worse |
| 2D equation | | | | | |
| Parabola 2D | 4.29 × 10⁻⁴ | 0.96392 | 0.02662 | −118.94819 | Better |
| equation | | | | | |
| Cosine | 1.06 × 10⁻² | 0.10970 | 0.64639 | 92.76439 | Bad |
| equation | | | | | |
| Extreme 2D | 1.06 × 10⁻³ | 0.91120 | 0.06553 | −58.59156 | Worse |
| equation | | | | | |
| Logistic Cum | 1.03 × 10⁻³ | 0.91337 | 0.06496 | −57.17690 | Worse |
| equation | | | | | |
| Fourier 2D | 2.24 × 10⁻³ | 0.81202 | 0.13424 | −14.54444 | Worse |
| equation | | | | | |
| Lorentz 2D | 1.41 × 10⁻³ | 0.88130 | 0.08901 | −36.07343 | Worse |
| equation | | | | | |

In table 2, RCS represents the weighted chi-squared test coefficient representative of a sum of squares of differences between data points and corresponding points of the fitting function. Origin is minimized by an iterative method, and the closer the value thereof to 0, the better. R² represents an adjusted coefficient of determination, and the closer the value thereof to 1, the better the fitting result. RSS represents a residual sum of squares, and the smaller the value thereof, the better the fitting result. The AIC represents the Akaike information criterion based on a maximum entropy principle, and the smaller the value thereof, the better the fitting result.

By contrast, the best goodness of fit is achieved when the damage model is established by selecting the Gaussian function (Gausscum equation). The relationship equation regarding the damage variable D, a deviatoric stress q, and a confining pressure σ₃ is fitted by using Origin software:

$$D = - 0.6294 + 0.4658675 [ 1 + erf \{ \frac{{σ}_{3} - 9693.70444}{- 7750.99122 \sqrt{2}} \} ] [ 1 + erf \{ \frac{q + 182.08148}{612 \sqrt{2}} \} ] \qquad ( 39 )$$

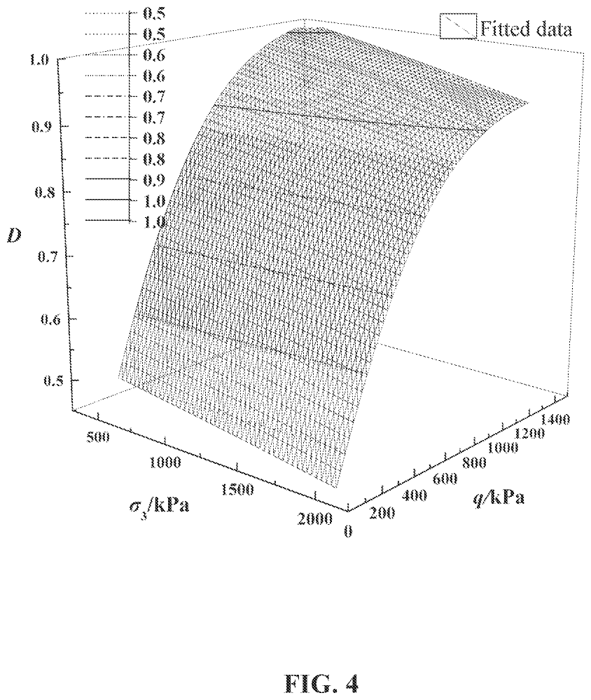

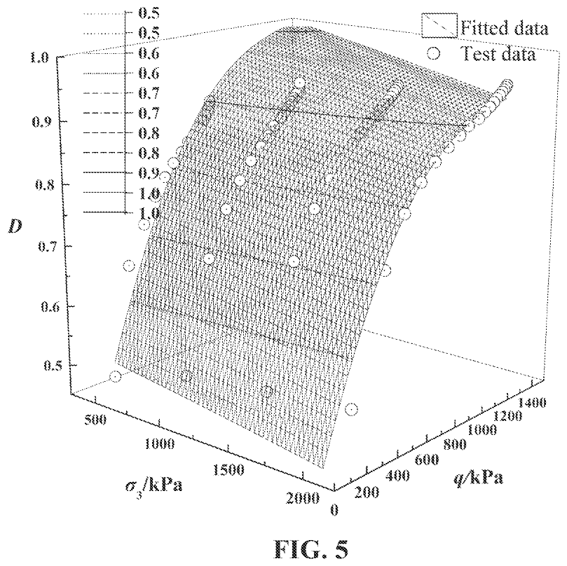

A relationship diagram of D-q-σ₃ fitted equation and a spatial curved surface is as shown in FIG. 4. A comparison diagram of D-q-σ₃ fitted data vs test data is as shown in FIG. 5, showing a high goodness of fit therebetween.

(5) The constitutive relationship equation is established. According to the formula (34), the following formulas may be derived under a conventional triaxial test condition:

$${ε}_{1} = \frac{{σ}_{1} - 2 {μσ}_{3}}{E ( 1 - D )} \qquad ( 40 - a )$$

$${ε}_{3} = \frac{{σ}_{3} - μ ( {σ}_{1} + {σ}_{3} )}{E ( 1 - D )} \qquad ( 40 - b )$$

This model is based on the conventional triaxial test with no consideration of an intermediate principal stress. The intermediate principal stress has little influence on soil strength, and the influence of the intermediate principal stress σ₂ may be neglected.

7. Damage model parameters are determined. A descending segment (softening) occurs after a stress-strain curve of mudstone under a certain confining pressure reaches an ultimate strength, and therefore, there is an extreme value. A stress and a strain corresponding to a peak point are recorded as _{σ}1c and _{ε}1c, respectively. The mudstone primitive strength _{F}c corresponding to the peak point is calculated according to the results of the triaxial test:

$${F}_{c} = \frac{[ α ( {σ}_{1 c} + 2 {σ}_{3} ) + \frac{{σ}_{1 c} - {σ}_{3}}{\sqrt{3}} ] E {ε}_{1 c}}{{σ}_{1 c} - 2 {μσ}_{3}} \qquad ( 41 )$$

The model parameters m and F₀ are calculated by the following formula:

$$m = \frac{1}{ln [ \frac{E {ε}_{1 c}}{{σ}_{1 c} - 2 {μσ}_{3}} ]} \qquad ( 42 )$$

$${F}_{0} = {F}_{c} {m}^{\frac{1}{m}} \qquad ( 43 )$$

Calculation results are shown in Table 3.

TABLE 3
Parameter Table of Mudstone Damage Model

| [symbol] /kPa | [symbol] /kPa | ε [symbol] | [symbol] /kPa | F [symbol] /kPa | m | F [symbol] /kPa |
|---|---|---|---|---|---|---|
| 500 | 745 | 0.0372 | 1245 | 1094 | 1.53 | 1445 |
| 1000 | 905 | 0.0423 | 190 [symbol] | 1648 | 1.30 | 2017 |
| 1500 | 1097 | 0.0429 | 2597 | 2334 | 1.16 | 2 [symbol] 53 |
| 2000 | 1 [symbol] 46 | 0.0461 | 3346 | [symbol] 161 | 1.06 | 3375 |

[symbol]

indicates data missing or illegible when filed

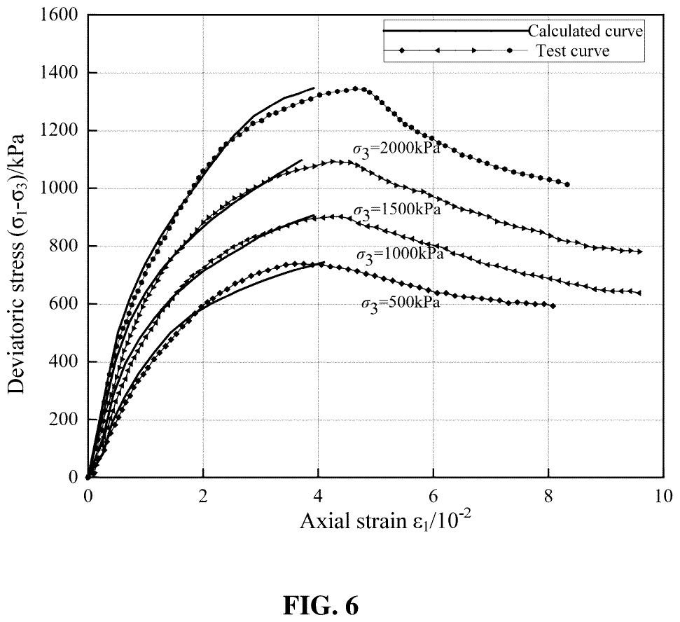

8. Verification is performed by comparison with the results of the triaxial test. The damage variable D is calculated by the formula (39) according to a loading grade of the triaxial test: a strain is then calculated from a stress by the formulas (40-a) and (40-b), and a comparison diagram of model calculation results with a curve of the triaxial test is plotted with an axial strain ε₁ as x-axis and a deviatoric stress (σ₁-σ₃) as y-axis, as shown in FIG. 6.

As shown in FIG. 6, at different confining pressure grades, the goodness of fit of the curved calculated according to the model disclosed herein with the test curve is high, and the accuracy of the novel damage constitutive model is verified.

9. The formula of the function relationship of a confining pressure σ₃ and a peak point σ_{1c} is fitted. By the triaxial test and damage analysis calculation, the relationship equation of a confining pressure σ₃ and a peak point σ_{1c} is obtained by fitting the function relationship therebetween using the method of least squares:

$${σ}_{1 c} = 635.8 + 1.176 {σ}_{3} + 8.9 \times {10}^{- 5} {σ}_{3}^{2} \qquad ( 44 )$$

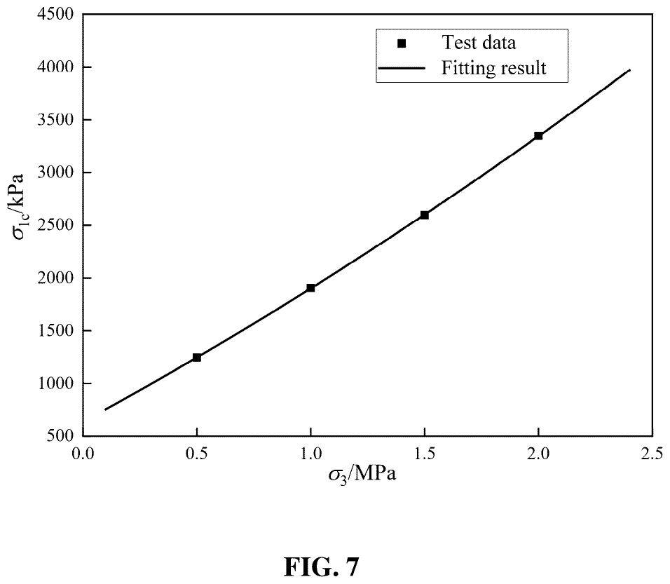

Fitting determination coefficient R² is 0.9999, indicating good correlation. The relationship curve is as shown in FIG. 7.

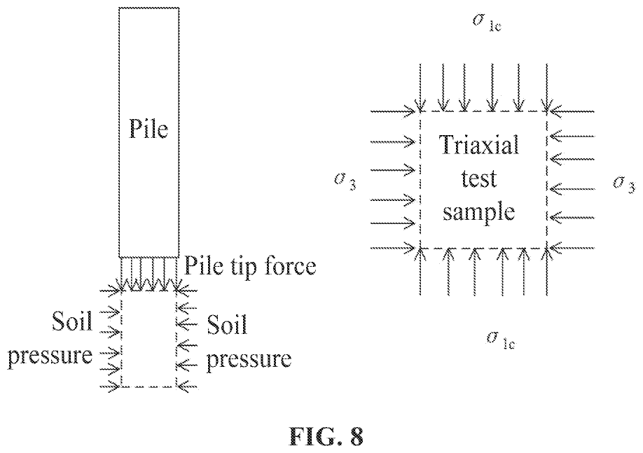

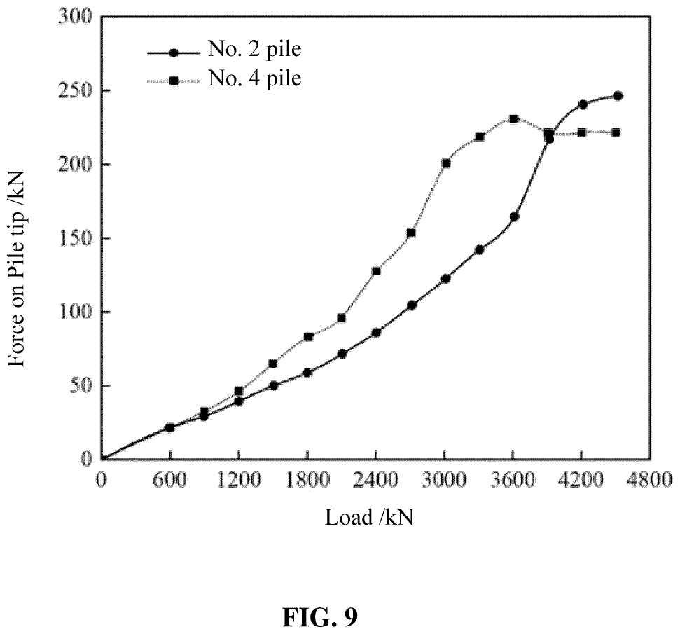

10. The pile tip resistance is calculated. The force condition of the mudstone at the pile tip of the test pile at the site of taking samples for the triaxial test herein is investigated. As shown in FIG. 8, the force state of the mudstone at the pile tip is similar to the force state of the mudstone in the triaxial test. The test pile on the site is a precast tubular pile having a pile diameter of 500 mm and a pile length (a depth into soil) of 15 m. In this case, the horizontal soil pressure of the mudstone at the pile tip is about 280 kPa, and this stress is substituted into the formula (44) as the confining pressure σ₃ to obtain the corresponding vertical destructive force σ_{1c}=972.1 kPa, which is equivalent to a concentrated load of 190.9 kN and compared with pile tip force results (FIG. 9) tested by sensors at test pile tips on the site. FIG. 9 illustrates two damaged piles in a static load test, and the pile tip forces are 253 kN and 221 kN, respectively, which are close to the model calculation results. The applicability of the model in analyzing the pile tip resistance is further verified.

### Example 2

In another typical embodiment of the present disclosure, as shown in FIG. 1 to FIG. 9, there is provided a system for predicting pile tip resistance based on a damage constitutive model of mudstone.

a parameter obtaining module configured to: obtain mechanical parameters of mudstone according to a triaxial compression test, and define a damage variable:
a fitting module configured to: according to a strain equivalence principle, establish a relationship of a stress and a strain with the damage variable, establish a damage evolution equation in combination with the mechanical parameters of the mudstone, and fit a relationship equation of the damage variable, a deviatoric stress, and a confining pressure;
a modeling module configured to: establish a damage constitutive model of mudstone, and calculate parameters of the damage constitutive model in combination with results of a triaxial test to obtain a correspondence between a confining pressure and a stress peak; and
a data output module configured to: obtain force data of the mudstone at a pile tip, calculate a confining pressure, and substitute the confining pressure into the correspondence to obtain pile tip resistance.

It will be understood that an operating method of the method for predicting pile tip resistance based on a damage constitutive model of mudstone is the same as the method for predicting pile tip resistance based on a damage constitutive model of mudstone provided in Example 1, and may be as shown in the detailed description in Example 1, which will not be described here redundantly.

The embodiments are described herein in a progressive manner. Each embodiment focuses on the difference from another embodiment, and the same and similar parts between the embodiments may refer to each other.

Specific examples are used herein for illustration of the principles and embodiments of the present disclosure. The description of the foregoing embodiments is used to help understand the method of the present disclosure and the core principles thereof. In addition, those of ordinary skill in the art can make various modifications in terms of specific embodiments and scope of application in accordance with the teachings of the present disclosure. In conclusion, the contents of the present description shall not be construed as limitations to the present disclosure.

What is claimed is:

1. A method for predicting pile tip resistance based on a damage constitutive model of mudstone, comprising:
obtaining mechanical parameters of mudstone according to a triaxial compression test, and defining a damage variable;
according to a strain equivalence principle, establishing a relationship of a stress and a strain with the damage variable, establishing a damage evolution equation in combination with the mechanical parameters of the mudstone, and fitting a relationship equation of the damage variable, a deviatoric stress, and a confining pressure;
establishing a damage constitutive model of mudstone, and calculating parameters of the damage constitutive model in combination with results of the triaxial test to obtain a correspondence between a confining pressure and a stress peak; and
obtaining force data of the mudstone at a pile tip, and substituting the force data as a confining pressure into the correspondence to obtain pile tip resistance.

2. The method for predicting pile tip resistance based on a damage constitutive model of mudstone according to claim 1, further comprising:
calculating an elasticity modulus and a shear strength indicator to obtain the mechanical parameters of the mudstone.

3. The method for predicting pile tip resistance based on a damage constitutive model of mudstone according to claim 1, further comprising:
discretizing a material into a plurality of primitives, wherein the damage variable is a ratio of a cross-sectional area of damage after the material is damaged to an initial cross-sectional area of the mudstone, and a primitive strength is assumed to comply with Weibull distribution.

4. The method for predicting pile tip resistance based on a damage constitutive model of mudstone according to claim 3, specifically comprising: calculating the damage variable based on data of a conventional triaxial test, and fitting the relationship equation of the damage variable, a deviatoric stress, and a confining pressure.

5. The method for predicting pile tip resistance based on a damage constitutive model of mudstone according to claim 1, specifically comprising: obtaining, according to the strain equivalence principle, a mudstone damage stress-strain relationship based on Weibull distribution as the damage constitutive model of mudstone, and optimizing the damage constitutive model of mudstone.

6. The method for predicting pile tip resistance based on a damage constitutive model of mudstone according to claim 1, wherein the calculating parameters of the damage constitutive model comprises the following steps:
recording a stress, a strain, and a confining pressure corresponding to a peak point after a stress-strain curve of mudstone under a confining pressure reaches an ultimate strength; and
calculating a mudstone primitive strength corresponding to the peak point based on the results of the triaxial test, and calculating the parameters of the damage constitutive model.

7. The method for predicting pile tip resistance based on a damage constitutive model of mudstone according to claim 6, further comprising:
substituting the obtained parameters of the damage constitutive model into the damage constitutive model, calculating the damage variable and the strain according to a loading grade of the triaxial test, and comparing the calculated damage variable and strain with the results of the triaxial test for verification.

8. The method for predicting pile tip resistance based on a damage constitutive model of mudstone according to claim 6, specifically comprising: fitting a function relationship between a confining pressure and a peak point by means of the triaxial test and damage analysis calculation to obtain the correspondence between a confining pressure and a stress peak.

9. The method for predicting pile tip resistance based on a damage constitutive model of mudstone according to claim 1, specifically comprising: obtaining pile parameters and a unit weight of a rock-soil body, and substituting a horizontal soil pressure of the mudstone at the pile tip as a confining pressure into the correspondence to obtain a corresponding vertical destructive force which is equivalent to a concentrated load as the pile tip resistance.

10. A system for predicting pile tip resistance based on a damage constitutive model of mudstone, comprising:
a parameter obtaining module configured to: obtain mechanical parameters of mudstone according to a triaxial compression test, and define a damage variable;
a fitting module configured to: according to a strain equivalence principle, establish a relationship of a stress and a strain with the damage variable, establish a damage evolution equation in combination with the mechanical parameters of the mudstone, and fit a relationship equation of the damage variable, a deviatoric stress, and a confining pressure;
a modeling module configured to: establish a damage constitutive model of mudstone, and calculate parameters of the damage constitutive model in combination with results of a triaxial test to obtain a correspondence between a confining pressure and a stress peak; and
a data output module configured to: obtain force data of the mudstone at a pile tip, and substitute the force data as a confining pressure into the correspondence to obtain pile tip resistance.
